(19) United States
(12) Patent Application Publication
(10) Pub. No.: US 2020/0237440 A1
(43) Pub. Date: Jul. 30, 2020

(54) Methods for Deflecting Catheters

(71) Applicant: EXIMO MEDICAL LTD, Rehovot (IL)

(72) Inventors: Yoel ZABAR, Nes Ziona (IL); Ilan BEN OREN, Modiin (IL); Oren Meshulam STERN, Shilo (IL); Shay GRANOT, Gan Shomron (IL)

(73) Assignee: EXIMO MEDICAL LTD, Rehovot (IL)

(21) Appl. No.: 16/839,523

(22) Filed: Apr. 3, 2020

Related U.S. Application Data
(63) Continuation of application No. 15/308,103, filed on Nov. 1, 2016.
(60) Provisional application No. 61/990,142, filed on May 8, 2014.
(60) Provisional application No. 62/102,125, filed on Jan. 12, 2015.

(51) Int. Cl. A61B 18/24; A61M 25/00; A61M 25/01; A61B 90/00

(52) U.S. Cl. CPC A61B 18/245; A61M 25/0054; A61M 25/0051; A61M 25/0158; A61B 90/37; A61B 2018/00601; A61M 25/0155; A61M 25/0147; A61M 25/0141; A61M 25/0133; A61M 25/0138

(57) ABSTRACT

New devices and methods for deflecting a catheter progressing within a lumen, into a preferred direction, typically in order to accomplish ablative removal of obstructive material within that lumen without the danger of uncontrolled catheter deflection risking perforation of the lumen. The catheter may ride on a guide wire, or it may be free riding down the lumen, limited by the passages available in the obstructive material, and generating its own passage by debulking the material within the lumen. The types of deflection required may be radial or lateral. A number of novel configurations are described, including improvements to the slotted wall catheter, by selection of the shape, spacing and location of the slots. Other implementations include a catheter with a novel spring configuration, which can release itself from a situation in which the catheter becomes stuck when widening an initial narrow bore in an obstructed vessel.

### FIELD OF THE INVENTION

The present invention relates to the field of catheter deflection, especially for use in debulking tissue from a lumen such as is performed in atherectomy.

### BACKGROUND

In lead extraction procedures, adhesion of the leads is often formed, especially in curved sections of the blood vessel, as shown FIG. 1a. When the electrode separation procedure is performed, there is a risk of perforation of the vein by the catheter, and in severe cases even death of the patients. Rates of 2% and even higher are reported using active dilators. Another need for deflecting the tip of a catheter is to achieve safe entry into the heart, where the catheter tip may impact valves, and when there maybe more than one lead in the same vein, such that maneuverability to the catheter is important to improve safety and efficacy.

In atherectomy and in stent restenosis applications, there are a number of challenges which need to be addressed when using cylindrical catheters:

(i) the need to deal with lesions that do not have radial symmetry, being non-concentric, and to by-pass local obstacles;

(ii) limitations of catheter profile size, which complicates situations where lumen openings that are larger than the catheter profile are required;

(iii) the maneuverability of the catheter tip in the region of vessel junctions;

(iv) positioning the tip in a required orientation when a guide wire cannot be used as a rail to guides the catheter, such as in Chronic Total Occlusions (CTO), where the guide wire cannot penetrate the blockage;

(v) the need to effectively collect debris and debulked material through the catheter lumen when the catheter lumen is significantly larger than the guidewire thickness.

Another example of a field in which there is a need for deflection of a catheter tip is in the debulking of material in the prostate, in management of BPH—Benign Prostatic Hyperplasia wherein creation of a lumen larger than the catheter diameter is of interest.

A number of prior art catheters exist, including hybrid catheters such as those described in US published patent application Nos. 2014/0031800 and 2014/0052114, and in International published patent application No. WO/2014/118738, each having common inventors with the present application.

The disclosures of each of the publications mentioned in this section and in other sections of the specification, are hereby incorporated by reference, each in its entirety.

### SUMMARY

The present disclosure describes new exemplary devices and methods for deflecting a catheter progressing within a lumen in a preferred direction, typically in order to accomplish ablative removal of obstructive material within that lumen. The catheter may ride on a guide wire, or it may be free riding down the lumen, limited by the passages available in the obstructive material, and generating its own passage by debulking the material within the lumen. The types of deflection required may be radial or lateral, but an important feature of the devices and methods described is that the tip of the catheter, where the laser emission all the surgical scalpel performs the ablation or cutting action, should not diverge significantly from its parallel orientation relative to the walls of the vessel, since such deflection may cause the tip of the lumen to perforate the walls of the vessel.

A number of novel configurations are described, including improvements to the slotted wall catheter, such that its orientation is better controlled so that the catheter does not perforate the walls of the blood vessel in which it is operating. This is done by careful selection of the shape, spacing and location of the slots. Other implementations include catheters which are able to release themselves from the common situation in which after a narrow bore has been cleared for the catheter, and the catheter is moved laterally to widen that bore, the edges of the widen bore may trap the catheter rendering it difficult to move backwards or forwards because of the danger of causing perforation of the vessel. A novel spring controlled configuration is described which enables the physician to release such a stuck catheter.

Other configurations described in this disclosure enable a guidewire-less annular catheter to efficiently debulk a large area of an obstructed vessel, by using a side deflection elements in order to stabilize the catheter without the assistance of a guide wire. The side deflection elements may be balloons, or mechanical structures which divert the catheter as required. Such mechanical structures or balloons may also be used in a novel method whereby an implanted lead stuck to the inner wall of the blood vessel by extraneous tissue growth, may be released even on sharp bends in the blood vessel, without the danger of perforating the vessel.

There is thus provided in accordance with an exemplary implementation of the devices described in this disclosure, a deflectable catheter comprising:

(i) an inner tube and an outer tube connected at their distal end,

(ii) an operating handle connected to the outer tube at its proximal end,

(iii) a spring attached at one end to the inner tube at its proximal end, and at its other end to an anchoring element, and

(iv) a spring loaded catch assembly attached to the handle, adapted to restrict the anchoring element from moving distally,

wherein the spring enables the movement of the inner tube in a distal direction, thus reducing the level of bending of the catheter.

Another implementation describes a method of performing catheter entry into an obstructed vessel, comprising:

(i) providing a composite catheter having a deflection feature and a debulking working head,

(ii) inserting the catheter into the vessel to generate a single entry path in the obstructed vessel,

(iii) withdrawing the catheter and using its deflection feature, moving the catheter radially aside by generating a double bend in the catheter,

(iv) drilling a second laterally shifted entry path, contiguous with the first entry path,

(v) advancing the catheter into the vessel to enlarge the single entry path, until a laterally located obstruction of material in the vessel prevents further advancement of the deflected catheter,

(vi) operating the deflection feature to reduce the level of bending of the catheter until it passes the laterally located obstruction of material in the obstructed vessel, and

(vii) continuing to advance the catheter into the obstructed vessel.

Additionally, there is also proposed as another exemplary implementation, a deflectable catheter comprising:

(i) an inner tube disposed inside an outer tube, the tubes being rigidly connected at their distal ends, at least one of the inner and outer tubes having:
(a) a first section near the distal end of the catheter, the first section having increased flexibility on one sector of the wall of the tube, and
(b) a second section proximal to the first section, having increased flexibility on a second sector of the wall of the tube, the second sector being disposed in a circumferential location generally opposite to that of the first sector of the wall of the tube,

wherein the increased flexibility of the first section increases towards the distal end of the first section, and the increased flexibility of the second section increases towards the proximal end of the second section,

In such a catheter, the sections having increased flexibility may be such that application of a differential tension between the inner and outer tubes results in a bending of the catheter at those sections. In that case, the increase of the increased flexibility of the first section towards the distal end of the first section, and the increase of the increased flexibility of the second section towards the proximal end of the second section should be such that the distal end of the catheter remains essentially parallel to its original direction. Additionally, according to yet another exemplary device, the sectors of increased flexibility may comprise a series of circumferential slots cut in part of the wall of at least one of the inner and outer tubes. Accordingly, the series of circumferential slots of the first section may be closer to each other at the distal end, and the circumferential slots of second section may be closer to each other at the proximal end. Additionally, the circumferential slots of the first section may be wider at the distal end of the first section, and the circumferential slots of the second section may be wider at the proximal end of the second section. Furthermore, the circumferential slots of the first section may be longer circumferentially at the distal end of the first section, and the circumferential slots of second section may be longer circumferentially at the proximal end of the second section.

In any of these last mentioned catheters described in this disclosure, the sectors of increased flexibility may advantageously comprise sections of the walls of the tubes having different thicknesses or being constructed of different materials. Also, the inner tube may be constructed of stiffened material, and it may advantageously be itself a catheter that includes at least one optical fiber. Any of these catheters may include flexible capillaries in order to inject saline from the proximal end to the distal end of the capillaries. Furthermore, in any of these implementations incorporating slots, the first section may advantageously be at distance of at least 10 mm from the distal tip of the outer tube. Additionally, the distance between the first section and the second section may be more than 10 mm.

Another example implementation can involve a method of extracting a lead from a blood vessel having a bend, utilizing any of the deflectable catheters described in this disclosure, comprising:

(i) inserting the deflectable catheter into the blood vessel,

(ii) determining when the deflectable catheter has reached the bend in the blood vessel,

(iii) aligning deflectable catheter so that a section of the circumferential slots is directed at the wall of the blood vessel at the outer radius of the bend, and

(iv) activating the deflecting mechanism such that the tip of the deflectable catheter negotiates the bend.

Additional implementations may involve a deflectable tubular catheter, comprising:

(i) a mechanical protrusion element stowed in the distal end region of the tubular catheter such that it does not protrude significantly from its outer radial bounds,

(ii) an adjustable activating mechanism which can deploy the protrusion element radially outwards of the tubular catheter, and can pull the protrusion element back within the outer radial bounds of the tubular catheter, the adjustable activating mechanism being connected to the proximal end of the catheter, such that it is operable by longitudinal motion from there, and

(iii) and wherein deployment of the protrusion element against a wall of the lumen causes the catheter to move away from the wall.

In such a deflectable tubular catheter the mechanical protrusion element may be a flexible spring tongue connected at one end to a tubular outer element of the tubular catheter, and the second end of the flexible spring tongue can be moved axially by the adjustable activating mechanism, such that the flexible spring tongue bends radially outwards. Alternatively, the mechanical protrusion element may a pre-shaped element made of shape memory alloy, and the activating mechanism may then be an outer tube that pushes the pre-shaped element back into its conformal configuration. According to yet another configuration, the protrusion lobe may comprise a flexible spring tongue attached at its proximal end to a tube incorporated into the tubular catheter, and at its distal end connected to the distal end of the catheter, such that proximal motion of the catheter relative to the tube causes the anchor points of the flexible spring tongue to move towards each other, thereby causing the flexible spring tongue to bend radially outwards. The mechanical protrusion element and the adjustable activating mechanism should be sufficiently flexible not to impair the insertion procedures of the catheter through a meandering lumen. In any of these protrusion element implementations, the protrusion element may be coated with a silicon layer.

Yet other implementations perform a method of extracting a lead from a blood vessel having a bend, utilizing any of the deflectable catheters described in this disclosure, comprising:

(i) inserting the deflectable catheter into the blood vessel,

(ii) determining when the deflectable catheter has reached the bend in the blood vessel,

(iii) aligning the deflectable catheter so that its protrusion element is directed at the wall of the blood vessel at the outer radius of the bend, and

(iv) deploying the protrusion element such that the tip of the deflectable catheter negotiates the bend.

According to yet another exemplary implementation of the devices of this disclosure, there is provided a system for debulking material from the inside of a lumen, comprising,

(i) a first annular catheter having annular walls with a plurality of fiber optical emitters disposed therewithin, and

(ii) a second tubular catheter having at least one fiber optical emitter disposed therein, and having a diameter substantially smaller than the diameter of the first annular catheter, the second tubular catheter being installed inside the annular space within the first annular catheter, and attached off-axially so that it has a common wall with the first annular catheter,

wherein the second tubular catheter protrudes forward from the first annular catheter. In such a system, the forward protrusion of the second tubular catheter should enable it to prepare an opening bore in any material inside the lumen, such that the first annular catheter can be directed down the lumen. In either of these implementations, the second tubular catheter protrudes forward from the first annular catheter only if deployed from within the first annular catheter, and the second tubular catheter may be in contact with at least one of the annular walls of the first annular catheter.

Further example implementations involve a system for debulking material from the inside of a lumen, comprising,

(i) a guidewire passing along the central region of the lumen, adapted to provide a path along which the catheter traverses the lumen,

(ii) an annular catheter whose annular hollow center has an inner dimension substantially larger than the diameter of the guidewire, and

(iii) a plurality of fiber optical emitters disposed inside the annular catheter,

wherein the inner dimension of the annular hollow center of the catheter, is substantially larger than the diameter of the guidewire, and wherein the catheter comprises at least one deflecting mechanism configured to move the annular catheter radially relative to the guidewire. In this system, the at least one deflecting mechanism may comprise a plurality of inflatable balloons disposed outside of the catheter, such that controlled inflation of one or more of the balloons enables the catheter to move radially relative to the guidewire. In either case, the motion of the catheter radially relative to the guidewire may be adapted to enable the plurality of fiber optical emitters to ablate material from the inside of the lumen from different regions of the inner wall of the lumen. Furthermore, the balloons may be attached to an outer tube and the catheter can slide axially within the outer tube.

### BRIEF DESCRIPTION OF THE DRAWINGS

The present invention will be understood and appreciated more fully from the following detailed description, taken in conjunction with the drawings in which:

FIGS. 1A and 1B show a novel annular catheter and its method of use, which enables efficient debulking and removal of the debulked material from a partially occluded blood vessel;

FIGS. 2A to 2C illustrate a further implementation of the catheter devices of the present disclosure in which two separate tubes are used to enable the composite catheter to perform debulking of tissue within a lumen in a single insertion;

FIGS. 3A to 3E illustrate a steered catheter device using a mechanical side protrusion lobe in order to steer the catheter;

FIGS. 4A and 4B illustrate an application for lead extraction in a curved blood vessel, using any of the deflecting catheters shown in this disclosure;

FIGS. 5A to 5D illustrate further implementations of a composite double tubed catheter, illustrating how a controlled bending operation can be achieved by use of slots formed over a part of the circumferences of the inner and/or outer tubes of a composite tube catheter;

FIGS. 6A to 6C illustrate schematically a method for generating a clear opening in an obstructed vessel by sequentially entering and deflecting the catheter in order to gradually open a clear passageway; and

FIGS. 7A to 7C illustrate schematically an apparatus and method by which the catheter can be extracted semi-automatically from a motion limiting situation such as that described in FIGS. 7A to 7C.

FIG. 8 illustrates an embodiment of the steerable catheter.

### DETAILED DESCRIPTION

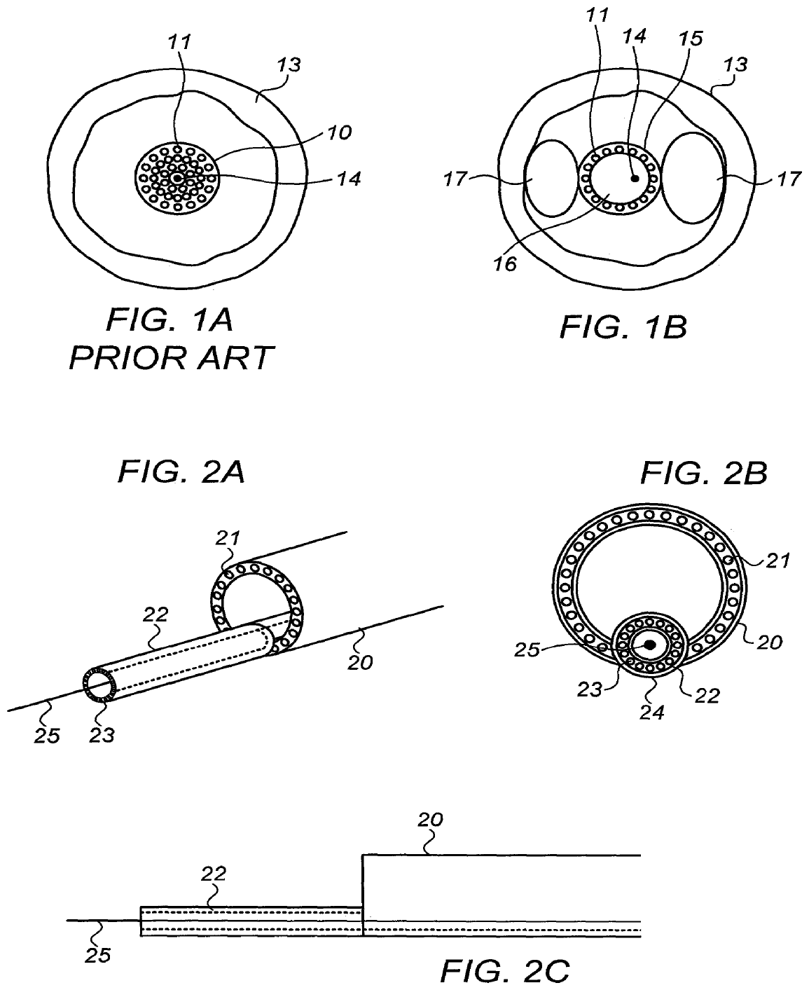

Reference is first made to FIG. 1A, which illustrates schematically a prior art situation in which a catheter 10, typically incorporating a plurality of light emitting optical fibers 11 for debulking material from the inner walls of a vessel 13, is progressing on a guidewire 14 which extends down the center of the vessel, and on which the catheter is riding. As the catheter proceeds, debulking material from the vessel walls, since the guidewire 14 diameter is of the same order as the size of the catheter lumen, and guides the catheter in a well-defined straight line path, the small inner bore of the catheter is unable to efficiently aspirate the debulked material, and debris, such as that remaining because of the spaces between the fibers and the catheter wall which do not contribute to the tissue ablation, can remain inside the vessel, with the danger that it can move downstream and block smaller arteries. In other implementations, the catheter is used to debulk material from other organs such the prostate in the management of Benign Prostatic Hyperplasia (BPH). In this application, an annular catheter can be used to debulk material that can be analyzed by histology means, to confirm that the patient does not have prostate cancer, as this may sometimes be present together with BPH. Using a catheter with deflection capabilities, enables the generation of large lumens in multiple paths of the catheter.

Reference is now made to FIG. 1B, which illustrates a method by which the debulked material can be efficiently removed from the distal work area of the catheter. As previously, the guide wire 14 passes down the center of the vessel 13 through the aspiration lumen of the catheter or alternatively in a separate lumen embedded inside the aspiration lumen. However the catheter 15 is of an annular design, having the fiber optical emitters 11 disposed within the annular cross-section of the catheter. A large central space 16 is thus generated within the annular catheter, allowing any debulked material to be aspirated therethrough from the distal end of the catheter. In some situations, the debulked material is not aspirated out of the catheter but remains locked inside. The position of the annular catheter 15 relative to the guide wire 14 can be defined by use of inflatable balloons 17 disposed on the sides of the annular catheter. By selectively deflating or inflating these balloons 17, the catheter can be made to move relative to the position of the guide wire 14, and this movement enables the catheter to debulk successively in different locations from within the vessel. Although only two balloons are shown in FIG. 1B, it is to be understood that balloons can be located at more than these locations around the annular catheter. The balloons 17 also keep the catheter stabilized and moving in a straight path down the vessel.

The deflection methods mentioned serve also to control tip positioning in cases that the position cannot rely on a guidewire, such as in Chronic Total Occlusions (CTO) and Benign Prostatic Hyperplasia (BPH) where a guidewire is not used or where catheters without a guidewire lumen are used.

Reference is now made to FIGS. 2A to 2C, which illustrate a further implementation of the catheter devices of the present disclosure. FIG. 2A is an isotropic view of this implementation, FIG. 2B is an end view and FIG. 2C is a side view. Two separate tubes are used to enable the composite catheter to perform debulking of tissue within a lumen in a single insertion. The separate tubes perform successive functions during the insertion process. The composite catheter device has a large diameter cylindrical annular outer tube 20, similar to that shown in FIG. 1B, with the annulus incorporating a plurality of fiber optical light energy emitters 21 for debulking the material within the lumen. In addition a smaller diameter cylindrical inner tube 22, also containing at least one fiber optical energy emitter 23, is attached to the wall of the annular outer tube, such that it is substantially off-center from the outer tube, and protrudes from the distal end of the outer tube. The two cylindrical tubes have a common wall 24. The guide wire 25, if used, can be threaded through the inner smaller diameter tube 22, and can exit at the end of this tube. In an alternative implementation, the smaller diameter cylindrical tube can have its distal end in the same plane as that of the outer cylindrical tube, using an ejection mechanism to push it forward so that it protrudes from the outer cylindrical tube.

This composite catheter is used in the following manner. As the composite catheter is advanced into the plaque laden lumen, the laser emission from the leading laterally positioned tube forms an opening in the tissue in advance of the large diameter tube following it. The formation of this initial bore along the guide wire enables the main debulking catheter tube to follow, and to remove the majority of the unwanted tissue, using the leading small diameter inner catheter tube to guide it forward over the guide wire. In order to enlarge the opened lumen, this procedure can be repeated several times at different angles, using the laterally positioned protruding tube as the axis of rotation. In some cases, the leading laterally positioned tube, can assist when the guidewire cannot readily pass through the plaque or calcified lesions. The optical fibers emitting the laser radiation can then assist in opening the way to the guidewire.

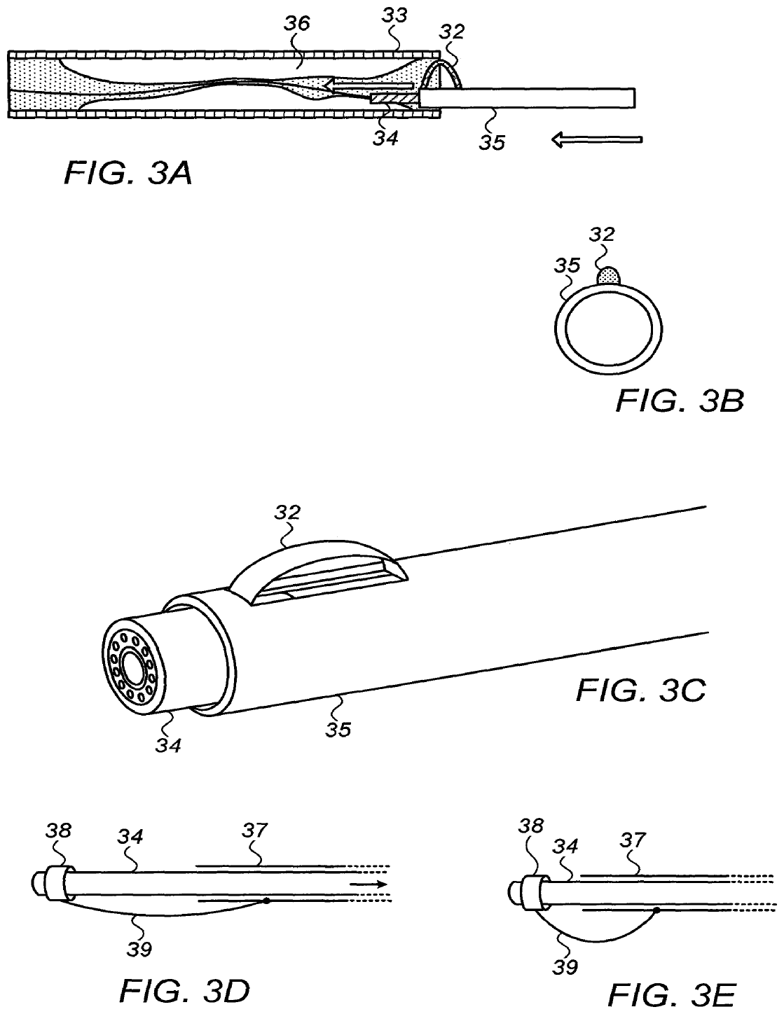

In order to divert or steer a catheter away from its current path, whether axial or not, it is possible to use an element projecting from the side of the catheter at its distal end, which pushes against the lumen wall and diverts the direction of motion of the catheter. Such a steering mode must be constructed so that the activating mechanism and the steering element lie within the outer bounds of the radius of the catheter, and furthermore, do not impair the flexibility of the catheter. Reference is now made to FIGS. 3A to 3E which illustrate one exemplary implementation of the catheter devices of the present disclosure, using a side protrusion lobe in order to steer the catheter. FIG. 3A is a cut-away representation of the catheter 34 in use in a lumen 33, FIG. 3B is an end view, while FIG. 3C is an isometric view of the catheter device. FIGS. 3D and 3E illustrate the component parts of the operating mechanism for the protrusion lobe, with FIG. 3D showing the non-deployed state and FIG. 3E showing the deployed state. The side protrusion lobe is deployed from the side of the catheter device at or near its distal end, such that when the contact surface of the lobe pushes against the side wall of the lumen, the catheter tip is steered in the opposite direction. The deployment of the lobe may be achieved by any suitable mechanism. The simple mechanism shown in FIGS. 3A to 3E uses a flexible spring element, though it is to be understood that any other mechanism which fulfills the above mentioned requirements will also be suitable, such as a tongue projection deployed by a spring, or an element made of a shape memory alloy such as Nitinol, either of which can be returned to their stowed position by means of a wire, or by means of a sliding sheath that pushes the element back into its conformal configuration.

Referring now to FIG. 3A, there is shown an application of the device entering an almost blocked lumen 33. In such an application, deployment of the steering lobe 32 out of the cover tube 35 surrounding the catheter, pushes the catheter 34 towards the side of the lumen, enabling it to clean out material 36 from the side of the lumen. The steering lobe 32 can either push the catheter against the wall of the vessel 33, as shown in FIG. 3A, or against the bore created by previous passage of the catheter 34. By rotating the catheter device and repeating the procedure several times, different circumferential parts of the accumulated material can be sequentially cleared out.

FIG. 3B shows an end view of the catheter showing the steering lobe deployed, while FIG. 3C shows the steering lobe showing through the outer sleeve of the catheter device.

Reference is now made to FIGS. 3D and 3E, which show one exemplary mechanism by which the steering lobe can be deployed using a tube and rod operating mechanism. FIG. 3D shows a flexible spring element 39, attached to one end of an outer sleeve 37, while the other end of the flexible spring element is attached by means of a collar 38 or by any other means to the catheter 34 which can slide within that sleeve. FIG. 3E shows the catheter 34 pulled relative to the outer sleeve 35 in the proximal direction, such that the flexible spring element 39 is pulled into its bent position, thereby deploying its central contact area perpendicularly away from the sleeve and catheter. This mechanism can be installed within the outer tube of a double tubed catheter such as is shown in FIG. 3C. The extent of the protrusion of the flexible spring element can be controlled by controlling the position of the catheter within the sleeve. The outer surfaces of the device, including the steering lobe, may be coated with a hydrophilic layer in order to reduce friction and/or a silicone layer in order to reduce the danger of damage to the lumen by the steering lobe pressure against its wall.

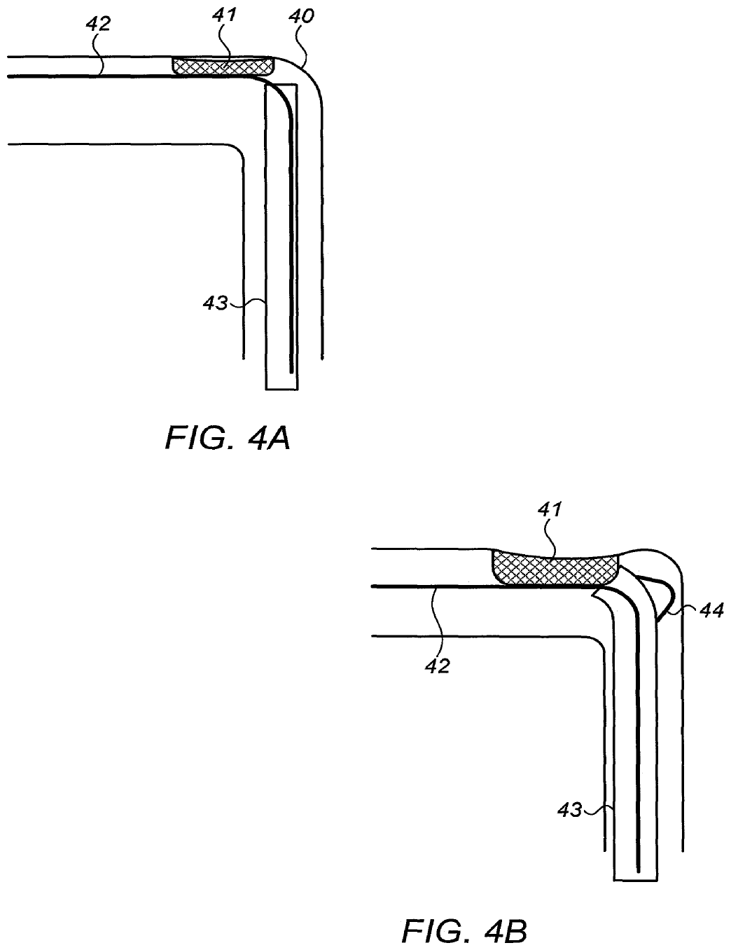

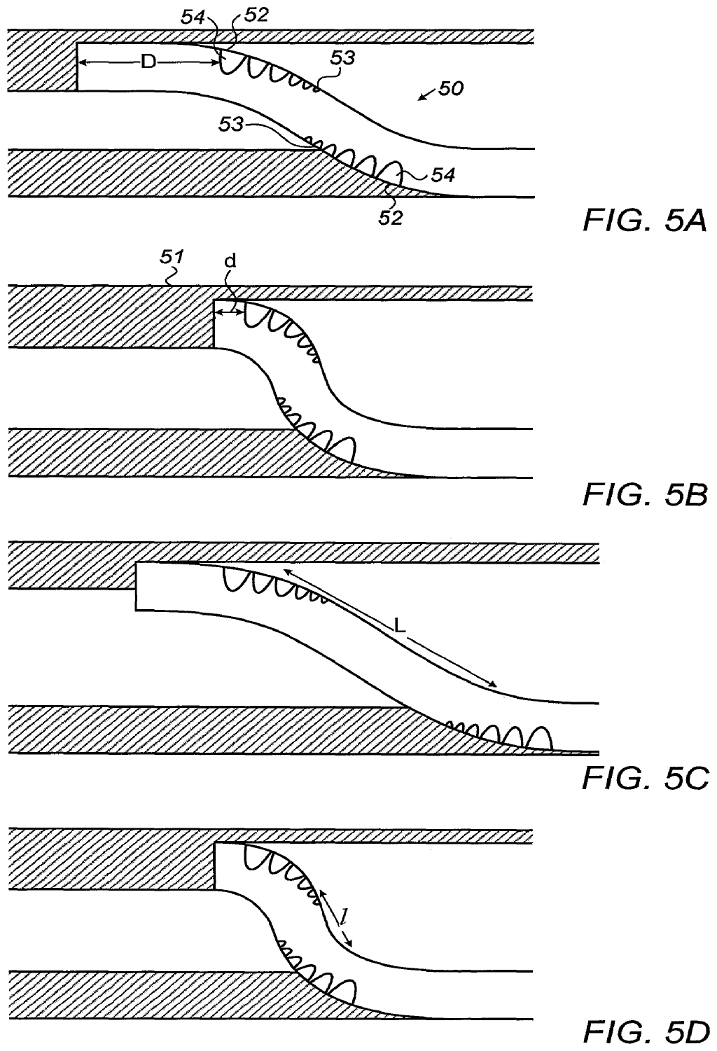

Reference is now made to FIGS. 4A and 4B which illustrate an application for lead extraction in a curved blood vessel, using any of the deflecting catheters shown in this disclosure. In the example shown in FIGS. 4A and 4B, the deflecting catheter of FIGS. 3A to 3E is used as an example of the procedure, but it should be understood that this is not meant to limit the invention, and that any steerable catheter can be effectively used, such as those with the graded slot implementation described hereinbelow in connection with FIGS. 5A to 5D. FIG. 4A shows a sharp bend 40 in a blood vessel, with some tissue 41 growing from the vascular wall and having attached itself to an electrode lead 42 passing down the blood vessel. In order to extract the lead safely, it is necessary to pass a catheter 43 over the lead, in order to detach it from the adherent tissue 41. In FIG. 4A, the catheter has reached the curve 40, and use of the deflecting catheter of FIGS. 3A to 3E enables the catheter to negotiate the curve safely without puncturing the blood vessel. This is shown in FIG. 4B, where the protruding lobe 44 has been deployed against the wall of the blood vessel, thereby forcing the catheter towards the center of the blood vessel and to distance itself from the wall, thus successfully negotiating the curve in the blood vessel.

Reference is now made to FIGS. 5A to 5D which illustrate further implementations of a composite double tubed catheter, illustrating how a controlled bending operation can be achieved. The inner and outer tubes are connected at the distal end. As is known in the prior art, either or both of the inner and outer walls of the tubes may have slots formed over a part of their circumferences, the slots providing greater bending flexibility to the wall on which they are situated. The wall side of the tube having the slots has less resistance to bending than the diametrically or circumferentially opposite side, and therefore, may generally become the outer side of any bend generated by linear tension applied to the slotted tube. When the slotted tubes are connected at their distal end, tension applied to the inner tube at the proximal end of the combined tube structure, causes the composite tube structure to bend, with the slots on the outer side of the bend. In order to generate a lateral shift of the composite tube structure, it is necessary to form two sets of slots, separated longitudinally from each other, each set of slots being on generally opposite sides of the tube. An S-shaped bend 50 is then formed in the structure, as shown in FIG. 5A, whose arrangement is known in the prior art. However the arrangements shown in the prior art may suffer from disadvantages in that unless the slots are designed in a predetermined manner to avoid such effects, application of compression on the inner tube relative to the outer tube may result in the distal end of the composite, S-shaped-bend tube, acquiring an outward angular orientation instead of a direction parallel to the axis of the catheter. This would involve danger that the catheter may perforate the outer wall 51 (labelled in FIG. 5B) of the vessel, especially when the deflection is of a catheter that emits strong laser radiation and/or manipulate a surgical blade. Therefore, it is important that the form and geometry of the slots be designed to prevent such radially outward resultant bending.

A number of alternative or cumulative features may be incorporated into the present implementation, in order to enable controlled bending, but without the distal end of the catheter acquiring an outward angular orientation. These features, which are not shown in previously proposed slotted connected tube structures, are shown clearly in FIGS. 5A to 5D.

One feature which can contribute to the control of the outward directed bending of the composite catheter is based on selection of the properties of the slots, their location relative to each other, and their location relative to the distal working end of the catheter. In order to implement control of the outward bending, the distal section of the outer tube which is intended to bend, has an arrangement of slots which provides more flexibility at its distal end than at its proximal end. This graduated flexibility can be generated by graduating the width, or the circumferential extent, or the closeness of the slots, such that the distal end of the curve-generating section is more flexible than the proximal end. As a result, there is less tendency for the distal end of the composite tube catheter to attain an outwardly directed orientation when its curve is generated by tension or pressure. In order to maintain bend symmetry, the proximal tube section which is intended to form the other part of the S-shaped bend, should have a symmetrically reversed flexibility profile to that of the distal section of the S-shaped bend, with the most flexible part being the proximal part of the slotted section. This is clearly shown in FIG. 5A, where the slots are shown being wider 54 at the outer ends 52 of the slotted sections than at their inner ends 53. It has been found that this arrangement contributes to preventing the distal end of the catheter from adopting an outward pointed orientation, which could result in perforation of the vessel wall by a laser and/or a blade and mechanical trauma of the inner vessel wall by a tip that stretches the vessel wall. The same can be relevant when a deflector is used to create lumens in other organs such as in the prostate in BPH.

A further feature which can be used to generate this graded flexibility within each section of increased flexibility is to arrange the slots to be closer together at the outer ends 52 of the slotted sections than at their inner ends 53. The closer together the slots, the greater the flexibility of the tube in that region. This feature is also illustrated in FIG. 5A. Additionally, by making the circumferential length of the slots longer, the flexibility of the tube in that region is increased. Therefore another method of achieving the graded flexibility in each section is by making the slots of greater length at the outer extremities 52 of the slotted sections than at their inner ends 53. This too is shown in FIGS. 5A to 5D.

This embodiment of generating higher flexibility to the slots at the distal end of the slot section relative to the proximal end of the slot section of the catheter can be used in lead extraction application wherein the catheter has to negotiate the curve of the Super Vena Cava (SVC) safely without puncturing the blood vessel. In this application, the catheter need only make a single bend with a single section of slotted tube, in order to bend away from the wall and around the curve in the vein. (This is different from the previously described applications where the catheter deflects itself laterally by means of 2 bends each with their own slot arrangement, in an S-shaped arrangement.) The higher flexibility at the distal end forces the catheter to bend inwards towards the center of the blood vessel and to distance itself from the wall, thus successfully negotiating the curve in the blood vessel.

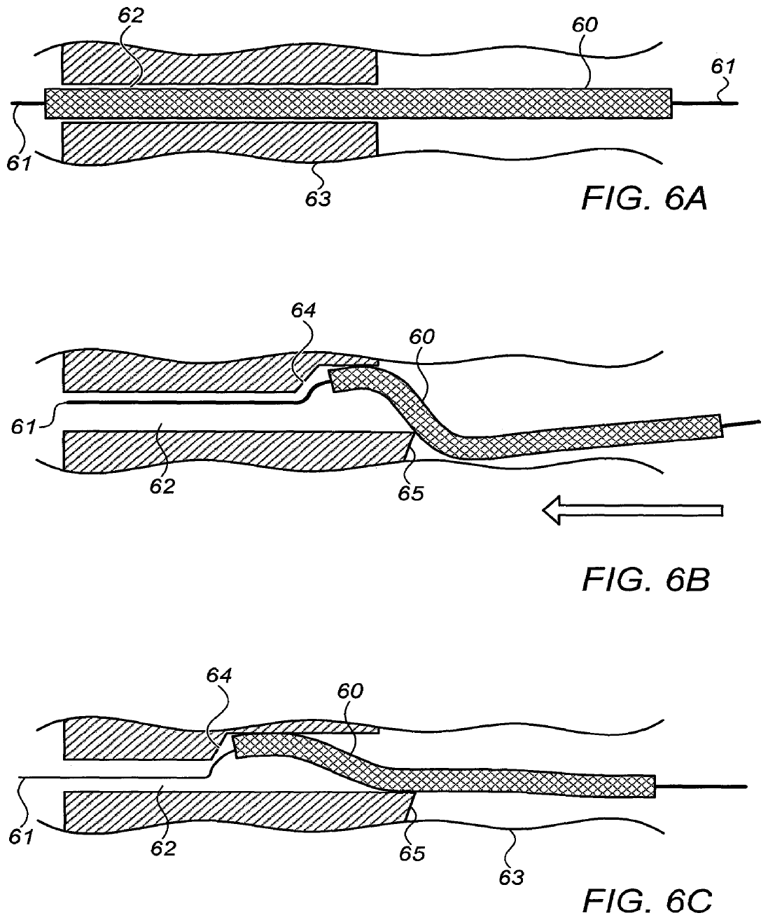

Additionally, the distal section of the slots may be positioned remotely at a distance D from the distal tip in order to achieve higher pushability of the distal end of the catheter, and in order to enable greater length of material debulking as illustrated in FIG. 5A. In addition, as observed in the situation of FIG. 6C, the well-spaced apart slot sections enables the catheter to generate an innermost opening of longer length, before the bend of the lumen impacts the corner shoulder of material of that innermost opening. In the catheters typically used for vascular treatment, a distance of 10 mm or more from the tip before the region of the slots is useful in this respect. This is in contrast to the catheter shown in FIG. 5B where the slots begin close to the distal end, at a distance d.

In some embodiments, the inner tube is made of a stiffened material in order to prevent the structure from bending outward. The inner tube can be a hybrid laser catheter, wherein its distal end contains optical fibers, blade that is made of stiffed material such us stainless steel, and glue that holds the whole structure.

Reference is now made to FIG. 5C which shows an implementation in which the two sets of slots, distal and proximal, are spaced apart by a distance “L”, which is selected to ensure that the bends have a sufficiently gentle gradient that optical fibers incorporated within the catheter will not be damaged. This distancing also enables the debulked material to be aspirated easily. Furthermore, the gradual bending helps the catheter to slide inside the lumen created by previous paths. In the catheters typically used for vascular treatment, a distance of 10 mm or more between the regions of the slots is useful in this respect. In some embodiments the length of section with slots is extended to 50-100 mm to enable a longer bending length and smaller angles and radius.

The implementation shown in FIG. 5C is in contrast to what is shown in FIG. 5D, where the slotted sections having a smaller distance “I” between them, where I<L, resulting in a much more acute bend with its associated potential problems.

In some embodiments the deflecting tube is covered with a flexible layer to facilitate sliding and prevent material getting into the slots. In some embodiments the cover tube is coated with hydrophilic coating.

In some embodiments the catheter includes flexible capillaries in order to inject saline from the proximal end to the distal end of the capillaries in order to prevent trauma to the vessel walls from interaction of the laser with the contrast media or the blood.

It is to be understood that the provision of flexibility in one circumferential section of the wall of the tubes by means of slots is only one method by which this flexibility can be achieved, and that the invention is not intended to be limited to the use of slots. The same selective circumferential or diametric flexibility can be achieved by having a tube of varying circumferential thickness, or of different materials in different circumferential sectors of the tube wall.

Reference is now made to FIGS. 6A to 6C, which illustrate schematically a method for generating a clear opening in an obstructed vessel by sequentially entering and deflecting the catheter in order to gradually open a clear passageway starting with a single entry. In FIG. 6A, the deflectable catheter 60 is shown riding on the guidewire 61, and has generated a single straight passageway 62 through the obstruction in the vessel 63. At this point, it is necessary to enlarge the diameter of the clear passage, and this is illustrated in FIG. 6B. The catheter 60 has been withdrawn and diverted laterally relative to the vessel axis by any of the methods or devices described in this application, for example, by pulling the catheter relative to an outer tube with slots, as shown in FIGS. 5A to 5D above, resulting in an “S-shaped” bent form, as shown in FIG. 6B. This diversion laterally shifts the distal tip of the catheter, so that it can then be pushed back into the obstruction in the vessel in a radially shifted position from the initially opened passageway 62, such that its working distal end is abutted against a section 64 of the obstructive material. The distal end of the catheter can now ablate more of the material to be removed, thereby enlarging the initial passageway already formed.

However, as the catheter moves forward, deepening the enlarged passageway, its progress may be stopped by its bent edge becoming wedged against another shoulder 65 of the remaining blockage material, situated on the opposite side of the vessel to that at which the catheter is now operating. In order to escape from this situation, the deflection needs to be reduced, as shown in FIG. 6C, enabling the lower bend profile of the catheter to pass the shoulder 65 of the blockage material, and to enter the lumen passage that was created by previous paths, so as to allow the distal end of the catheter to continue enlarging the passageway down the vessel. This reduction in the deflection shown in FIG. 6C can be achieved by retracting the deflecting action used to generate the deflection needed to begin the second enlarging entry shown in FIG. 6B. In such a situation, a simple semi-automatic process can be used, which operates in accordance with the forces applied to the catheter. This can be applied, for example, by a spring that expands and contracts according to the force generated on the catheter by the blockage or impediment, and/or blood vessel or other lumens such as in BPH.

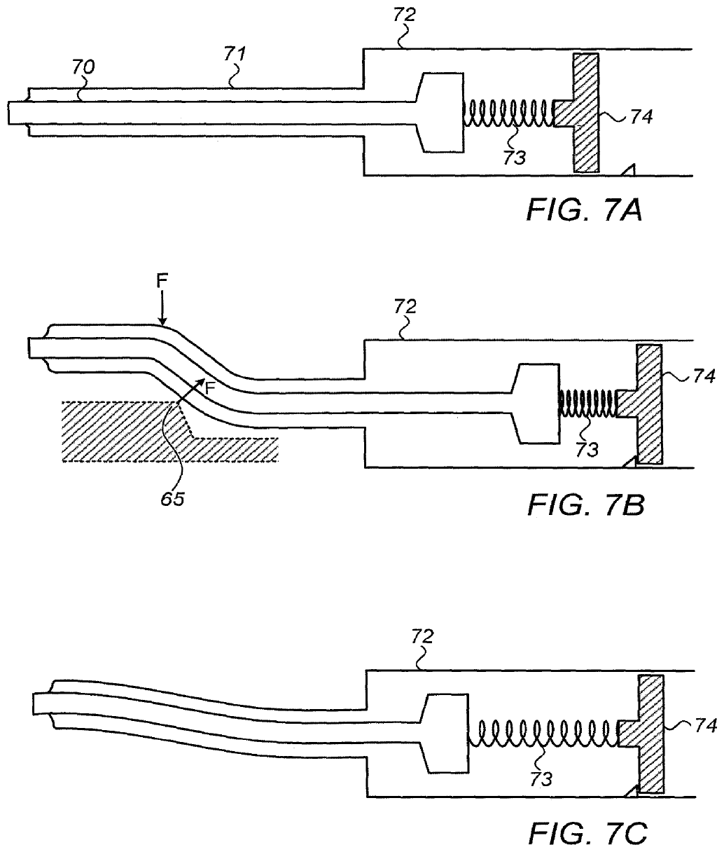

Reference is now made to FIGS. 7A to 7C, which illustrate schematically such an apparatus and method by which the catheter can be extracted semi-automatically from such a motion limiting situation. FIGS. 7A to 7C are drawn to be exactly equivalent to FIGS. 6A to 6C above, so that the mechanism can be readily followed. FIG. 7A shows the structure of the catheter, having an inner tube 70 and an outer tube 71 connected only at their distal end. The proximal end of the outer tube 71 terminates in a handle 72 which the physician uses to manipulate the catheter within the blood vessel being treated. The inner tube 70 is connected at its proximal end to a base knob 74 by means of a spring 73. During normal use, as the catheter bends during its progress down the blood vessel being treated, the inner tube 70 can move freely in an axial direction relative to the outer tube 71, and the unloaded spring 73 transfers this to-and-fro motion directly to the base knob 74.

The base knob 74 can also be used by the physician in order to divert the tip of the catheter. This can be done by holding the handle 72 stationary, such that the catheter does not move axially, and by pulling proximally on the base knob 74. Since the inner and outer tubes are connected only at their distal end, and because of, for instance, a slotted structure in the outer tube to provide flexibility, this results in bending of the catheter in an S-shape along its length. This bending then results in deflection of the tip radially from its original position. This situation is shown in FIG. 7B. Conversely, if the catheter is already in a bent situation, pushing the base knob 74 distally will result in reduction of the bent condition.

The semi-automatic freeing action is engendered by an additional structure within the handle 72. A set of pins 75 is incorporated within the handle proximally to the handgrip in its free position, and these pins are spring-biased and shaped with a chamfered or sloping distal edge such that the base knob 74 can move proximally past them, but having passed them, cannot move distally back. Therefore, when the catheter undergoes a deflection beyond a certain predetermined level, the handgrip moves proximally past the pins 75, which thus block the base knob 74 from moving distally again. It is to be understood that similar methods other than the use of spring-biased pins, may also be used to accomplish this feature. At this point the deflection of the catheter cannot be controlled by the physician by manipulation of the handgrip 74, but it is controlled by extension or compression of the spring 73.

Therefore, referring again to FIG. 7B, and relating FIG. 7B back to the situation in FIG. 6B, when the catheter gets into a trapped position against a shoulder obstruction 65 of material and cannot proceed down the blood vessel, the following process occurs:

1. With abutment of the catheter against the shoulder 65, a force F is applied to the catheter wall at the point of contact with the abutment. This force is in a direction normal to the wall of the catheter, and as such, will tend to decrease the bending of the catheter if conditions allow it to. In addition, and as is apparent from the situation shown in FIG. 6B, similarly forces F may be applied by the lumen walls on the catheter, mostly from the top direction (in the reference frame of the drawing) but possibly also from the bottom, and this also has a major vector part which is perpendicular to the catheter axis.

2. Because the catheter is slightly flexible, even when in its trapped position, the reaction forces F applied on the outer wall, with or without perpendicular forces by the lumen wall, will slightly reduce the bend in the wall, to the extent that the flexibility of the catheter allows it to.

3. Any straightening of the catheter results in the inner tube moving distally, this being the reverse process to the method of generating a deflection by pulling the inner tube proximally.

4. This distal motion of the inner tube causes the spring 73 to be extended, because its proximal end is anchored by the base knob 74 behind the spring biased pins. The straighter the catheter becomes, the more distal is the position of the inner tube.

5. The decreased deflection thus enables the catheter to be reinserted into the lumen that was created by the catheter.

This situation is shown in FIG. 7C, where the catheter has straightened out substantially, while extending the spring 73 in the process, and this straightened catheter can now pass the obstruction shoulder 65 and proceed with its motion distally down the obstructed vessel.

If the outer tube is made of a thin metallic material, such as stainless steel or nitinol, the spring may alternatively be embedded in the outer tube by laser processing, which can engrave a spring on the walls, instead of the spring that is connected to the inner tube.

The spring may alternatively be connected to pull/push wires that are used to deflect a catheter as known in the art of deflecting catheters. The operating wire or wires are generally attached to the distal end of the catheter, and deflection of the catheter is actuated by means of a proximal handle device held by the physician, manipulation of which pulls the operating wire or wires. In the same way as a double tube catheter described in this implementation can get stuck by becoming wedged between obstructions in the vessel while clearing the passageways, the wire guided catheter can also become stuck. The solution described in FIGS. 7A to 7C can therefore also be applied to the wire guided catheter, in which case, the spring or springs which enable the release mechanism to operate, may be attached in the line of the operating wire or wires, conveniently between the wire or wires and the operating handle device. Though it can be made simpler than the device shown in FIGS. 7A to 7C, the operating concept is the same, and the invention described in this disclosure is not meant to be limited to the case of the double tube catheter guidance.

The above described implementation relates to a catheter wherein the deflection is made by pulling the inner tube in the proximal direction relative to the outer tube. If the deflection properties are generated by use of a slotted structure, as described herein, the bending may be achieved by pushing the inner tube in the distal direction relative to the outer tube. In that case the spring should be undercompression rather than extended.

Axial force dependent deflection may also be controlled by using feedback from imaging cameras or monitoring sensors which can detect the presence of the blockage. For example, feedback from light reflected back out of the catheter fibers can help the physician to determine where to position the catheter, since the signal reflected from a passageway generated in a previous passage is expected to be lower than the signal when the catheter faces the blockage or vessel. Alternately intravascular ultrasound (IVUS) or Internal imaging can be used.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

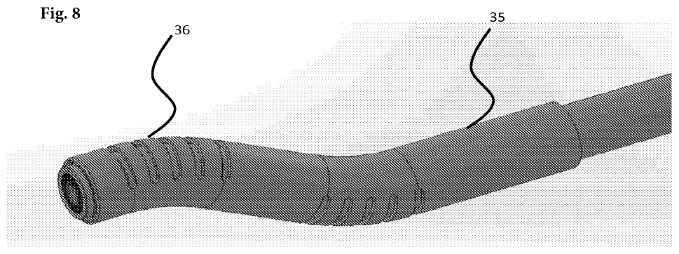

FIG. 8 is another embodiment, wherein slots 36 are created in the outer tube 35, and the longitudinal movement creates a deflection of the tube. Slots 36 in one side of the tube will perform a radial deflection, while slots in two sides of the tube will perform an “S” shape deflection as illustrated in FIG. 8. In some embodiments, the catheter is used as the inner tube and is connected to the outer tube at the distal tip either by fixation or by a bracket and notch mechanism. In some embodiments the slots are created in the inner tube. In some embodiments a steerable catheter is used in order to deflect the catheter. The steerable sheath is maneuverable by pulling a wire that is embedded in the sheath and the tension is deflecting the sheath tip.

1.-20. (canceled)

21. A method comprising the steps of:
placing an atherectomy device into a vessel, the atherectomy device comprising an atherectomy device distal end and a plurality of fiber optical emitters, the atherectomy device distal end comprising a deflection element, the deflection element comprising a plurality of slots formed over at least a part of a circumference of the deflection element, and the atherectomy device distal end comprising a straight configuration and an off-centered configuration;
advancing the atherectomy device toward a treatment site;
deflecting the atherectomy device distal end from the straight configuration to the off-centered configuration such that the deflection element laterally shifts the plurality of fiber optical emitters and the atherectomy device distal end;
debulking a target tissue near the treatment site.

22. The method of claim 21, wherein the atherectomy device further comprises an aspiration lumen.

23. The method of claim 22, further comprising the steps of:
advancing the atherectomy device over a guidewire;
aspirating the debulked target tissue through the aspiration lumen; and
withdrawing the atherectomy device from the treatment site.

24. The method of claim 21, wherein the step of deflecting the atherectomy device distal end further comprises applying tension to the deflection element.

25. The method of claim 21, wherein the step of deflecting the atherectomy device distal end further comprises the deflection element to form a substantially S-shape.

26. The method of claim 21, further comprising the steps of:
reducing potential for perforation of the vessel; and
reducing potential of mechanical trauma to an inner wall of the vessel.

27. The method of claim 21, further comprising the step of:
deflecting the atherectomy device distal end from the off-centered configuration to the straight configuration to such that the deflection element laterally shifts the plurality of fiber optical emitters and the atherectomy device distal end.

28. The method of claim 24, wherein the atherectomy device further comprises a handle, and further comprising the step of:
manipulating the handle by a user results in applying tension to the deflection element.

29. The method of claim 21, wherein the step of debulking the target tissue near the treatment site comprises emitting light energy from the plurality of fiber optical emitters.

30. A method comprising the steps:
placing a treatment device into a vessel, the treatment device comprising a treatment device distal end, a plurality of fiber optical emitters, and a deflection element on the treatment device distal end, the deflection element comprising a plurality of slots formed over at least a part of a circumference of the deflection element, and the treatment device distal end comprising a straight configuration and an off-centered configuration;
advancing the treatment device over a guidewire toward a treatment site;
deflecting the treatment device distal end from the straight configuration to the off-centered configuration such that the deflection element laterally shifts the plurality of fiber optical emitters and the treatment device distal end;
debulking a target tissue near the treatment site;
advancing the treatment device distal end into the target tissue without the assistance of the guidewire thereby forming an open passageway through the target tissue.

31. The method of claim 30, further comprising the step of:
advancing the guidewire through the open passageway of the target tissue formed by the treatment device.

32. The method of claim 30, wherein the target tissue is a chronic total occlusion.

33. The method of claim 30, further comprising the step of:
reducing potential for perforation of the vessel.

34. The method of claim 30, wherein the step of deflecting the treatment device distal end further comprises the deflection element to form a substantially S-shape.

35. A method comprising the steps:
placing a device into a vessel, the device comprising a distal end, an aspiration lumen, a plurality of optical fibers, and a distal tip deflection element, the distal tip deflection element comprising a plurality of slots formed over at least a part of a circumference of the distal tip deflection element, and the device distal end comprising a straight configuration and an off-centered configuration;
advancing the device toward a treatment site over a guidewire;
deflecting the device distal end from the straight configuration to the off-centered configuration such that the distal tip deflection element laterally shifts the plurality of optical fibers and the device distal end;
debulking a target tissue near the treatment site; and
aspirating the debulked target tissue through the aspiration lumen.

36. The method of claim 35, wherein the step of deflecting the device distal end further comprises distal tip deflection element to form a substantially S-shape.

37. The method of claim 36, wherein the step of debulking the target tissue near the treatment site comprises emitting light energy from the plurality of fiber optical emitters.

38. The method of claim 35, further comprising the step of:
reducing potential for perforation of the vessel.

39. The method of claim 35, wherein the target tissue may include any of the following: a chronic total occlusion, a calcified lesion, an in-stent restenosis, or a plaque material.

40. The method of claim 35, wherein the device further comprises a handle, and further comprising the step of:
manipulating the handle by a user resulting in tension being applied to the distal tip deflection element.
